(19) United States
(12) Patent Application Publication
(10) Pub. No.: US 2004/0267295 A1
(43) Pub. Date: Dec. 30, 2004

(54) Reduced shear force microkeratome blade assembly

(76) Inventor: William J. Neubert, Ballwin, MO (US)

Correspondence Address: Michael L. Smith, Bausch & Lomb Incorporated, One Bausch & Lomb Place, Rochester, NY, 14604-2701, US

(21) Appl. No.: 10/609,816

(22) Filed: Jun. 30, 2003

(51) Int. Cl. A61F 9/00; A61B 17/32; B27B 13/02; B27B 33/14; B23D 57/00

(52) U.S. Cl. 606/166; 606/167

(57) ABSTRACT

A microkeratome blade assembly 20 includes a cutting blade 22 having a forward cutting edge 24. A blade holder 26 is attached to the blade 22. At least one indentation 30 is formed in a front surface 29 of the blade holder 26 for reducing shear force between the blade holder 26 and a microkeratome cutting head 38.

### BACKGROUND OF THE INVENTION

1. Field of the Invention

The present invention is related to microkeratome cutting blade assemblies.

2. Description of Related Art

The use of microkeratome cutting blade assemblies is well known, such as that described in U.S. Pat. No. 6,051,009 to Hellenkamp, et al. which is herein incorporated in its entirety by reference. Typically, a microkeratome cutting blade assembly 10, such as shown in FIG. 1 of the prior art fits in within a receiving slot of a microkeratome cutting head assembly.

The cutting blade 12 is then oscillated by the microkeratome back and forth by a pin via a slot 14 in blade holder 16. In this way, cutting blade assembly 10 is moved across a subject eye by the microkeratome, a flap of corneal material is typically created, which flap will be resected during LASIK (laser-assisted in-situ keratomileusis) surgery.

The interaction and inter-workings between the microkeratome blade assembly 10 and a microkeratome cutting head, are critical to ensure that a consistent flap is created with a clean, even cut.

Because of the tight tolerances required to be maintained to ensure that a proper corneal flap thickness is achieved, it is common that the microkeratome cutting blade assembly fits snuggly within a microkeratome cutting head assembly to prevent microkeratome cutting blade assembly 10 from being moved around during operation of the microkeratome and to precisely locate the blade assembly 10 within the cutting head. In addition, the cornea of the patient is typically lubricated with various lubricants including BSS (balanced-salt solution). These lubricants can wick themselves into the gap between the cutting head assembly and the cutting blade assembly, and it is believed may actually increase the shear force resistance of the cutting blade assembly within the cutting head assembly to oscillation back and forth within the cutting head assembly as required during operation of the microkeratome.

Therefore, it is desirable to have an improved microkeratome cutting blade assembly which would reduce any shear forces and provide an outlet for lubricants that are wicked into the microkeratome cutting head/cutting blade assembly interface.

### BRIEF DESCRIPTION OF THE DRAWINGS

FIG. 1 is a side elevation of a prior art microkeratome cutting blade assembly;

FIG. 2 is a side elevation of a microkeratome cutting blade assembly in accordance with the present invention;

FIG. 3 is a front elevation of FIG. 2;

FIG. 4 is a side elevation view of a microkeratome cutting blade assembly in accordance with the present invention held within a compatible cutting head assembly;

FIG. 5 is a side elevation view of an alternate embodiment in accordance with the present invention; and

FIG. 6 is yet another alternate embodiment in accordance with the present invention.

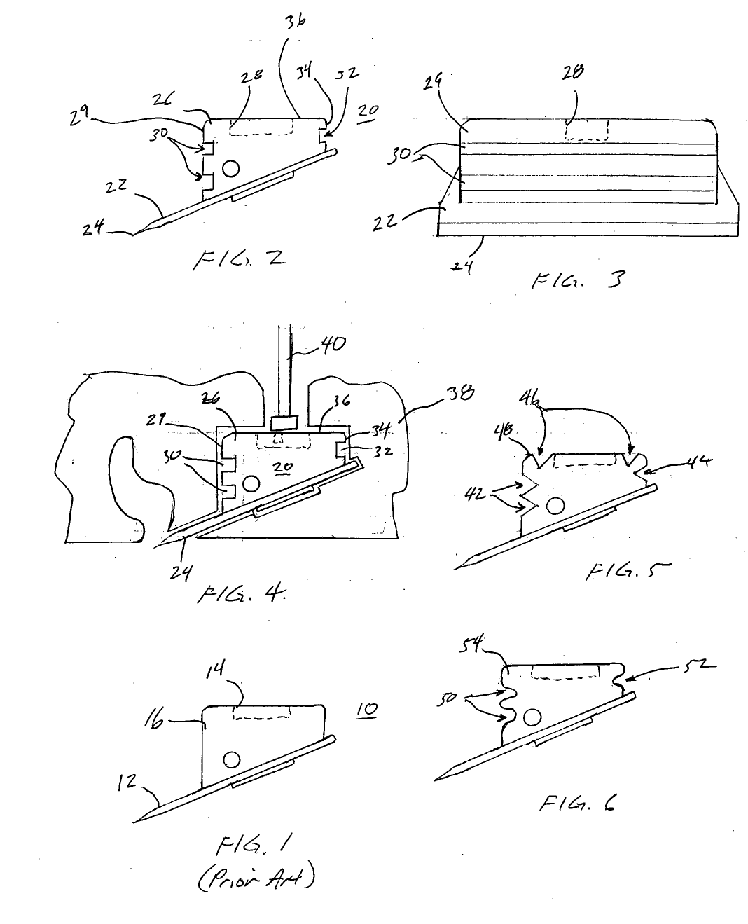

### DETAILED DESCRIPTION

FIG. 2 shows a side elevation view of a microkeratome cutting blade assembly 20 in accordance with the present invention. Blade assembly 20 includes a blade 22 having a forward cutting edge 24 and a blade holder 26 attached to the blade 22. Blade holder 26 is structured to be oscillated by a microkeratome (not shown) via slot 28. Preferably at least one indentation 30 is formed in a front surface of blade holder 26 for reducing shear force between the blade holder 26 and a microkeratome cutting head as described further below.

Blade holder 26 may be attached to blade 22 by any of several methods, such as cold staking, heat staking, adhesives, or any other means to securely attach blade holder 26 to blade 22. Blade 22 may be metal, such as that formed from razor-blade stock or blade 22 may be formed of ceramic material or other such materials that can be formed with a sufficiently sharp cutting edge 24 to cut a cornea in preparation for LASIK surgery. Likewise, blade holder 26 may be at least partially formed of ceramic material and as such, may actually be integral to blade 22.

In addition, blade assembly 20 also includes an indentation 32 in a rear surface 34. Front surface 29 preferably includes multiple indentations 30 which are most preferably grooves as shown which extend across the front surface 29, as clearly shown in FIG. 3. In addition to grooves 30 and 32, top surface 36 may also include grooves extending the length of such top surface, as described in more detail below.

With respect to FIG. 4, cutting blade assembly 20 is held within a cutting head assembly 38. Typically, tight tolerances are held between the cutting head assembly and front surface 29, rear surface 34, and top surface 36. As such, blade assemblies 20 which are at the outer limits of the manufacturing specifications may fit rather snuggly within cutting head assembly 38. Thus, the provisions of grooves 30 and 32 reduces the shear force between the cutting head assembly 38 and the contacting surfaces of the blade holder 26 and also wick away and provide a channel for moisture and lubricants that are used on a patient's eye during the cutting of a corneal flap. It is believed by providing such grooves 30 and 32 that the blade assembly 20 will be more freely oscillated by oscillation pin 40 during use of the microkeratome.

FIG. 5 shows an alternate embodiment of the present invention, wherein the grooves are V-shaped as shown at 42, 44, and 46. Grooves 46 are preferably formed in a top surface 48 to reduce the shear force at the intersection of top surface 48 and a cutting head assembly.

FIG. 6 shows yet another alternate embodiment having rounded grooves 50 and 52 formed in a blade holder 54. As can be seen from the various embodiments shown, the grooves can be of many geometrical shapes and lengths. That is the grooves do not need to necessarily span the length of the blade holders to reduce the shear force between the blade holder and a cutting head. In fact, the grooves do not need to be grooves but may rather be indentations which do not provide a straight channel but rather may be a more randomized pattern on the blade holder surfaces.

I claim:

1. A microkeratome cutting blade assembly comprising:
a cutting blade having a forward cutting edge;
a blade holder attached to the blade and structured to be oscillated by a microkeratome; and
at least one indentation in a front surface of the blade holder for reducing shear forces between the blade holder and a microkeratome cutting head.

2. The invention of claim 1, wherein the cutting blade assembly is structured to be oscillated from a top surface of the blade holder.

3. The invention of claim 1, wherein the cutting blade is formed of metal.

4. The invention of claim 1, wherein the cutting blade is formed of ceramic.

5. The invention of claim 4, wherein the blade holder is at least partially formed of ceramic.

6. The invention of claim 1, wherein the indentation is a groove extending across the front surface.

7. The invention of claim 6, wherein there are multiple grooves.

8. The invention of claim 1, further including an indentation in a rear surface of the blade holder.

9. The invention of claim 8, wherein the rear indentation is a groove extending across the rear surface.

10. A microkeratome cutting blade assembly comprising:
a cutting blade having a forward cutting edge;
a blade holder attached to the blade and structured to be oscillated by a microkeratome; and
at least one groove extending across a front surface of the blade holder and at least one groove extending across a rear surface of the blade holder for reducing shear force between the blade holder and a microkeratome cutting head.

11. The invention of claim 10, wherein the cutting blade assembly is structured to be oscillated from a top surface of the blade holder.

12. The invention of claim 10, wherein the cutting blade is formed of metal.

13. The invention of claim 10, wherein the cutting blade is formed of ceramic.

14. The invention of claim 13, wherein the blade holder is at least partially formed of ceramic.

15. The invention of claim 10 further including at least one groove extending across a top surface of the blade holder.
